(19) United States
(12) Patent Application Publication
(10) Pub. No.: US 2011/0171188 A1
(43) Pub. Date: Jul. 14, 2011

(54) NON-THERMAL PLASMA FOR WOUND TREATMENT AND ASSOCIATED APPARATUS AND METHOD

(75) Inventors: Gregor Morfill, Munich (DE); Bernd Steffes, Garching (DE); Tetsuji Shimizu, Garching (DE); Rene Pompl, Munich (DE); Tetiana Nosenko, Garching (DE); Wilhelm Stolz, Munich (DE); Georg Isbary, Munich (DE); Hans-Ulrich Schmidt, Pullach (DE)

(73) Assignee: Max-Planck-Gesellschaft zur Foerderung der Wissenschaften e.V, Munich (DE)

(21) Appl. No.: 13/060,006

(22) PCT Filed: Aug. 17, 2009
(86) PCT No.: PCT/EP2009/005957

§ 371 Date: Mar. 24, 2011

(30) Foreign Application Priority Data
Aug. 27, 2008 (EP) 08015146.7

(51) Int. Cl. A61K 38/43; A61K 33/00; A61K 33/24; A61K 33/38; A61K 33/34; A61K 33/32; A61K 33/30; A61K 33/26; A61K 33/22; A61K 33/14; A61K 33/18; A61K 33/06; A61K 33/04; A61K 38/00; A61K 31/045; A61K 31/02; A61K 31/01; A61K 47/00; A61P 17/02; A61B 18/00

(52) U.S. Cl. 424/94.1; 424/600; 424/617; 424/618; 424/619; 424/630; 424/639; 424/641; 424/646; 424/657; 424/661; 424/667; 424/682; 424/709; 424/723; 424/724; 514/1.1; 514/724; 514/759; 514/762; 514/769; 606/34

(57) ABSTRACT

The invention relates to a non-thermal plasma for treatment of a surface, particularly for the treatment of a wound (1), wherein the plasma comprises a partially ionized carrier gas and at least one additive, which preferably has a sterilizing effect on the treated surface and/or improves the healing of the wound (1). Further, the invention relates to a corresponding apparatus and method.

### FIELD OF THE INVENTION

The invention relates to a non-thermal plasma for treatment of a surface, particularly for the treatment of living tissue and especially for the treatment of wounds.

Further, the invention relates to an apparatus and a corresponding method for providing such a non-thermal plasma, particularly for the treatment of living tissue and especially for the treatment of wounds.

### BACKGROUND OF THE INVENTION

The use of non-thermal plasmas for the in-vivo sterilization of wounds is disclosed, for example, in WO 2007/031250 A1 and PCT/EP2008/003568.

However, it is desirable to improve the sterilizing effect of the plasma on wounds thereby improving the wound healing.

Further, reference is made to US 2007/029500 A1, US 2006/084158 A1, WO 2005/000363 A, WO 02/32332 A and US 2004/094400 A1.

### SUMMARY OF THE INVENTION

Therefore, it is a general object of the invention to improve the sterilizing effect of the plasma and the wound healing in a plasma therapy.

This object is achieved by a novel non-thermal plasma comprising at least one additive, which has a sterilizing effect and/or improves the healing of a wound. Therefore, the plasma according to the invention can be termed as a designer-plasma which is specifically designed for the treatment of biological tissue, e.g. wounds, skin, etc., while not harming healthy tissue.

The term sterilization means that the concentration of bacteria is reduced by the plasma, which encompasses a reduction by a factor of 10² (i.e. decontamination), 10⁴ (i.e. disinfection) or 10⁶ (i.e. sterilization).

The additive can be selected from a variety of substances including salts (e.g. sulfates, chlorides), metals, organic substances, inorganic substances and compounds or mixtures of the afore-mentioned substances. Other examples for the additive are biomolecules, proteins and enzymes.

Specifically, the additive can be selected from a group consisting of boron, bromine, thallium, silicon, iron, aluminium, silver, particularly colloidal silver, copper, zinc, manganese, ZnSO₄, K₂, MnO₄, FeSO₄, Ti₂SO₄, iodine, SiO₂, KMnO₄, zinc sulfate, copper-(I)-chloride or copper-(II)-chloride, silver nitrate, silver chloride, manganese-(II)-sulphate, (2-bromine-2-nitrovinyl)-benzole or compounds or mixtures of the afore-mentioned substances.

Other examples of additives are helium, neon, argon, krypton, xenon, nitric oxide, oxygen, hydrogen, sulfur hexafluoride, nitrous oxide, hexafluorethane, methane, carbon fluoride, fluoroform, carbon dioxide, ethanol, air, water or mixtures of these substances.

However, it is essential that the additive has a beneficial effect with regard to the plasma treatment. It is preferred that the additive has a beneficial effect on organic or living tissue. In other words, the additive is preferably health-improving. For example, the additive can be a substance which has a sterilizing effect and/or which improves the wound healing. Therefore, the additive is preferably bactericidal, fungicidal and/or antiviral. However, it is also possible that the additive improves the plasma generation or the plasma application.

It should further be noted that the non-thermal plasma according to the invention can comprise different additives with different properties. For example, a bactericide can be used as first additive and a fungicide can be used as a second additive.

It should further be noted that the additives can be gaseous, solid or liquid.

It should further be noted that the additive in the novel plasma can be a substance which can be activated compared with the starting material. Therefore, the plasma can comprise the additive either in an activated form or in an inactive form. In the further process, the additive can be activated.

It should further be noted that the additive in the plasma can be dissociated or non-dissociated.

Moreover, the additive can be a substance which can be coagulated due to thermal effects or for other reasons. Therefore, the plasma can comprise the substances in a coagulated form or in a non-coagulated form.

Further, the novel plasma can comprise the additive either in an ionized form or in a substantially non-ionized form.

The invention further comprises the novel use of the afore-mentioned non-thermal plasma for the treatment of wounds, living tissue or organic tissue.

Another field of application for the non-thermal plasma according to the invention is the sterilization of a natural or artificial body orifice of a human or animal body and/or for the sterilization of a medical instrument during insertion of the medical instrument through the body orifice into a lumen of the human or animal body, wherein the medical instrument is preferably a catheter. For example, the flow of the plasma can be directed to the body orifice in order to avoid an intrusion of bacteria or other pathogens through the body orifice. Further, the plasma can be directed onto the medical instrument (e.g. a catheter) during the insertion of the instrument into the body so that no pathogens are introduced into the body by the medical instrument.

Further, the plasma according to the invention can be used for the sterilization of transplants, e.g. skin, kidneys, livers, hearts or lungs.

Another field of application for the plasma according to the invention is the treatment of skin diseases or skin disorders.

Further, the plasma according to the invention can be used for the sterilization or treatment of a visceral cavity or lumen of a human or animal body, particularly for the sterilization of an oral cavity or an intestinal cavity.

Finally, the plasma according to the invention can be used for the manufacture of a medicine for the treatment of wounds or biological tissue. In this application, the plasma itself constitutes the medicine which can for example be used for the treatment of skin disorders or skin diseases wherein the wound healing is improved.

Moreover, the invention encompasses an apparatus for providing the afore-mentioned non-thermal plasma, particularly for the treatment of wounds.

The apparatus according to the invention comprises at least one carrier gas source which provides a carrier gas, e.g. argon or ambient air. However, the invention is not restricted to a specific type of carrier gas. Therefore, other types of carrier gases can be used, as well, e.g. helium or nitrogen.

It should further be noted that the novel plasma according to the invention can comprise a mixture of several different carrier gases. Therefore, the apparatus according to the invention can comprise several carrier gas sources providing different carrier gases which are mixed.

Further, the apparatus according to the invention comprises at least one plasma generator for ionizing the carrier gas which is provided by the carrier gas source, so that the plasma generator generates a non-thermal plasma. The plasma generator can be a conventional plasma generator as disclosed, for example, in WO 2007/031250 A1 and PCT/EP2008/003568. However, other types of plasma generators can be used, as well. Further, there can be several plasma generators which can be arranged in series or in parallel.

Moreover, the apparatus of the invention comprises at least one additive source providing the additive. For example, the additive source can be a simple gas cylinder containing the additive in a gaseous form.

Alternatively, the additive source can be a coating of an electrode arrangement in the plasma generator, wherein the coating consists of the additive so that the additive escapes from the coating into the carrier gas. In this embodiment, the plasma generator also forms a mixer which is mixing the additive and the carrier gas.

In another embodiment of the invention, the additive source is a component (e.g. a heated wire or a heatable silver ring), which is coated with the additive so that the additive escapes from the component during operation of the apparatus. For example, the additive can be extracted from the component by heating or sputtering the component. Further, the component may be a massive component consisting of the additive. In this embodiment, the afore-mentioned component forms a mixer mixing the additive and the carrier gas.

It should further be noted that the novel plasma according to the invention can comprise a mixture of several different additives. Therefore, the apparatus according to the invention can comprise several additive sources providing different additives which are mixed.

Finally, the apparatus according to the invention comprises a mixer which is mixing the additive with the non-ionized carrier gas and/or with the ionized plasma. For example, the mixer can be simply a junction of two conduits which are fed by the carrier gas on the one hand and by the additive on the other hand. However, the mixer can also be realized in other ways, which has already been mentioned above.

The mixer generally determines the ratio between the carrier gas and the additive, whereas the plasma generator determines the degree of ionization of the plasma, i.e. the percentage of ionized atoms or molecules. Therefore, the mixer is preferably adjustable in such a way that the ratio between the additive and the carrier gas can be adjusted. Further, the plasma generator is preferably adjustable in such a way that the degree of ionization (i.e. the percentage of ionized atoms or molecules) of the plasma can be adjusted.

In a first embodiment of the invention, the additive is mixed with the non-ionized carrier gas, i.e. before the ionization of the carrier gas. In this embodiment, the mixer is arranged upstream before the plasma generator and mixes the non-ionized carrier gas and the non-ionized additive, so that the plasma generator ionizes a mixture of the carrier gas and the additive.

In a second alternative, the mixer is mixing the additive with the ionized plasma, i.e. after the ionization of the carrier gas. In this alternative, the mixer is arranged down-stream after the plasma generator and mixes the ionized carrier gas provided by the plasma generator and the substantially non-ionized additive provided by the additive source.

In a third alternative embodiment, the mixer mixes the ionized carrier gas and the ionized additive. Therefore, the mixer is arranged downstream after the plasma generator(s). For example, there can be a first plasma generator for ionizing the carrier gas and a second additive for ionizing the additive. In such a case, the output of both plasma generators is connected to the mixer so that the mixer is arranged downstream after both plasma generators.

It should further be noted that several different additives can be mixed with the carrier gas and/or with the ionized plasma. Therefore, the apparatus according to the invention can comprise several additive sources for providing the different additives.

Further, several mixers can be provided for mixing the different additives with the non-ionized carrier gas and/or with the ionized plasma.

The plasma generator preferably comprises an electrode arrangement for electrically exciting the carrier gas and, possibly, the additive thereby generating the plasma as disclosed, for example, in WO 2007/031250 A1. Further, the apparatus preferably comprises a high-voltage generator which is connected to the electrode arrangement of the plasma generator.

However, other types of plasma generators are possible, as well. For example, the plasma can be produced by an antenna arrangement or by photo-ionization.

In one embodiment of the invention, the mixer is arranged upstream before the plasma generator so that the plasma generator receives a mixture of the carrier gas and the additive. However, the additive source provides the additive to the mixer discontinuously, so that there are additive-free time intervals during which no additive is provided to the plasma generator, and additive-containing time intervals during which the plasma generator is receiving the additive from the additive source. The discontinuous operation of the additive source can be realized, for example, by providing a controllable valve between the additive source and the plasma generator. In this embodiment, the plasma generator is preferably activated during the additive-free time intervals only, so that the additive is substantially not ionized within the plasma generator although the additive passes through the plasma generator.

In this embodiment, the apparatus preferably comprises a controller, which is controlling both the activation of the plasma generator and the gas flow from the additive source to the plasma generator in such a way that no additive is provided to the plasma generator during activation of the plasma generator.

However, it is alternatively possible that the controller controls the activation of the plasma generator and the gas flow from the additive source to the plasma generator in such a way that the mixing of the additive and the ionization within the plasma generator are temporally overlapping. In this embodiment, the degree of ionization (i.e. the percentage of the ionized atoms or molecules) of the additive is determined by the overlapping time-frame between the time period, in which the plasma generator is activated, on the one hand and the time period during which the additive is provided to the plasma generator, on the other hand. Therefore, it is possible to adjust the degree of ionization (i.e. the percentage of the ionized atoms or molecules) of the additive by adjusting the afore-mentioned overlapping time-frame.

In one embodiment of the invention, the afore-mentioned high-voltage generator of the plasma generator produces a pulse train consisting of pulses which are separated by gaps. This can be achieved by periodically switching the high-voltage generator on and off via a controller. Alternatively, the pulse train can be realized by a switch between the high-voltage generator and the plasma generator, wherein the switch is periodically opened and closed. In this embodiment, the additive can be provided to the plasma generator during the gaps only, so that the additive is substantially not ionized by the plasma generator although the additive passes through the plasma generator.

Further, the apparatus can comprise a UV shield (UV: ultraviolet radiation) which is arranged between the plasma generator and the treated object (e.g. a wound) so that any ultraviolet radiation emitted by the plasma generator is at least partially blocked by the UV shield. Therefore, no UV radiation or only a small fraction of the originally generated UV radiation reaches the treated object.

In this embodiment comprising a UV shield, the mixer can be arranged downstream behind the UV shield so that the additive is added to the plasma downstream behind the UV shield with the result that the additive is not affected by the UV radiation which is generated by the plasma generator.

In another embodiment, a catheter is provided for introducing the plasma through a natural or artificial body orifice into a lumen of a human or animal body. For example, the catheter can be introduced through the mouth into the gullet in order to sterilize the gullet, which might be helpful for the treatment of gullet cancer. In this case, the plasma is designed in such a way that it has a cytotoxic effect in order to inactivate malignant cells.

Finally, the invention also encompasses a method of treating a surface, particularly a wound, which is already apparent from the afore-mentioned description.

It should further be noted that the additive is preferably partially ionized, wherein the degree of ionization (i.e. the percentage of the ionized atoms or molecules) is above 1·10⁻⁹, 2·10⁻⁹, 5·10⁻⁹, 10⁻⁸, 2·10⁻⁸, 5·10⁻⁸, or 10⁻⁷ when measured in the plasma production region. Alternatively, the additive can be substantially not ionized, wherein the degree of ionization (i.e. the percentage of the ionized atoms or molecules) is below 10⁻¹⁵, 10⁻¹⁶, 10⁻¹⁷ or 10⁻¹⁸. It should be noted that the term partially means that there is a fraction of atoms and molecules that is ionized.

It should further be noted that the plasma according to the invention preferably comprises a gas temperature (i.e. the temperature of the atoms or molecules) below +100° C., +75° C., +50° C. or +40° C., when measured on the treated surface. Further, the pressure of the plasma preferably equals atmospheric pressure, wherein the pressure is preferably in the range of 800 hPa-1.200 hPa and more preferably in the range of 900 hPa-1.100 hPa, when measured on the treated surface. Moreover, the degree of ionization (i.e. the percentage of the ionized atoms or molecules) of the carrier gas is preferably above 1·10⁻⁹, 2·10⁻⁹, 5·10⁻⁹, 10⁻⁸, 2·10⁻⁸, 5·10⁻⁸ or 10⁻⁷ when measured in the plasma production region.

It should further be noted that the plasma can also be applied to the surface (e.g. a wound) in a special low pressure environment below 800 hPa.

The invention and its particular features and advantages will become more apparent from the following detailed description considered with reference to the accompanying drawings.

### BRIEF DESCRIPTION OF THE DRAWINGS

FIG. 1 is a schematical representation of an apparatus according to the invention for providing a non-thermal plasma containing an additive which is improving the wound healing,

FIG. 2 is a schematical representation of another embodiment of an apparatus for providing a non-thermal plasma for a wound treatment, wherein both the additive and the carrier gas are ionized and then mixed,

FIG. 3 is a schematical representation of another embodiment of an apparatus for providing a non-thermal plasma for wound treatment, wherein several different additives are mixed with the carrier gas,

FIG. 4 is a schematical representation of an outlet of a plasma source comprising a UV shield blocking ultraviolet radiation,

FIG. 5 is a graphical representation of a modification of the embodiment according to FIG. 4, wherein the additive is mixed with the plasma downstream behind the UV shield, and

FIG. 6 is a schematical representation of a catheter for introducing the non-thermal plasma into a lumen of a human body through a body orifice.

### DETAILED DESCRIPTION OF THE INVENTION

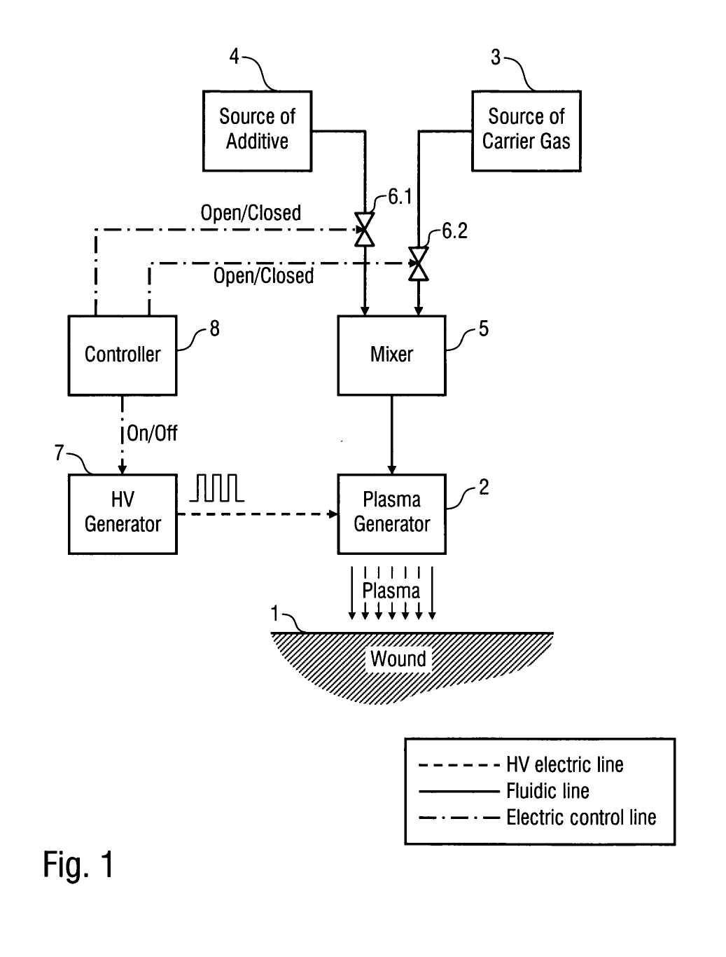

FIG. 1 shows a schematical representation of an apparatus for providing an improved non-thermal plasma for the treatment of a wound 1.

The apparatus comprises a plasma generator 2 which can be a conventional plasma generator as disclosed, for example, in WO 2007/031250 A1.

Further, the apparatus comprises a carrier gas source 3 providing a carrier gas, and an additive source 4 providing a gaseous additive which is improving the wound healing.

The additive source 4 is connected to a mixer 5 via a controllable valve 6.1 and the carrier gas source 3 is connected to the mixer 5 via a another controllable valve 6.2.

Therefore, the mixer 5 receives the non-ionized carrier gas (e.g. argon) from the carrier gas source 3 and the non-ionized additive from the additive source 4 and mixes these gases. Then, the mixer 5 provides the mixture of the carrier gas and the additive to the plasma generator 2 which ionizes both the carrier gas and the additive thereby generating the plasma.

The plasma generated by the plasma generator 2 is then applied to the wound 1 wherein the additive has a sterilizing effect on the wound 1 and improves the healing on the wound 1.

It should further be noted that the plasma generator 2 comprises an electrode arrangement for producing the plasma, wherein the electrode arrangement is connected to a high-voltage generator 7 which produces a pulse train consisting of pulses exciting the plasma and gaps between successive pulses. Therefore, the plasma generator 2 ionizes the mixture of the carrier gas and the additive discontinuously in an intermittent on/off-operation with intervals in which the plasma generator 2 is not activated.

Further, the apparatus comprises a controller 8 which controls the pulse train generated by the high-voltage generator 7 and the valves 6.1, 6.2 in such a way that no additive is provided during intervals in which the plasma generator 2 is activated by the high-voltage generator 7. However, the controller 8 opens the valve 6.1 in the intervals during successive pulses of the pulse train generated by the high-voltage generator 7, so that the additive is provided to the plasma generator 2 during the inactive intervals of the plasma generator 2 only. Therefore, the additive is not substantially ionized by the plasma generator 2 although the additive passes trough the plasma generator 2.

Further, the controller 8 controls the ratio between the carrier gas and the additive by controlling the valves 6.1, 6.2 accordingly.

It should further be noted that the high-voltage generator 2 and the valve 6.1 can be controlled in such a way that the active intervals of the plasma generator 2 and the open-intervals of the valve 6.1 are overlapping so that the additive is ionized during the overlapping time interval. Thus, the degree of ionization (i.e. the percentage of ionized atoms or molecules) can be adjusted by adjusting the overlapping time interval.

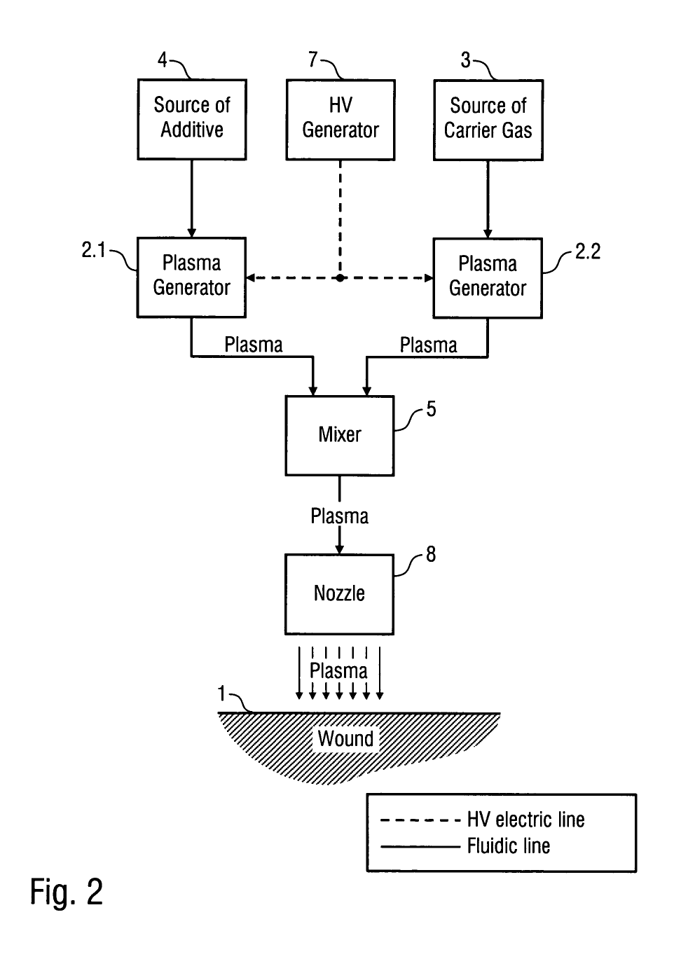

FIG. 2 shows a schematical representation of another embodiment of an apparatus for providing an improved non-thermal plasma. The embodiment of FIG. 2 is similar to the embodiment of FIG. 1 so that reference is made to the above description of FIG. 1. Further, the same reference numerals are used for corresponding parts and components.

One characteristic of this embodiment is that both the additive and the carrier gas are ionized separately. Therefore, there are two plasma generators 2.1, 2.2 for ionizing the additive and the carrier gas, respectively.

The plasma generators 2.1, 2.2 are connected to the mixer 5 which is mixing the ionized additive and the ionized carrier gas.

The mixer 5 is in turn connected to a nozzle 8 forming a plasma flow which is directed onto the wound 1 for improving the wound healing.

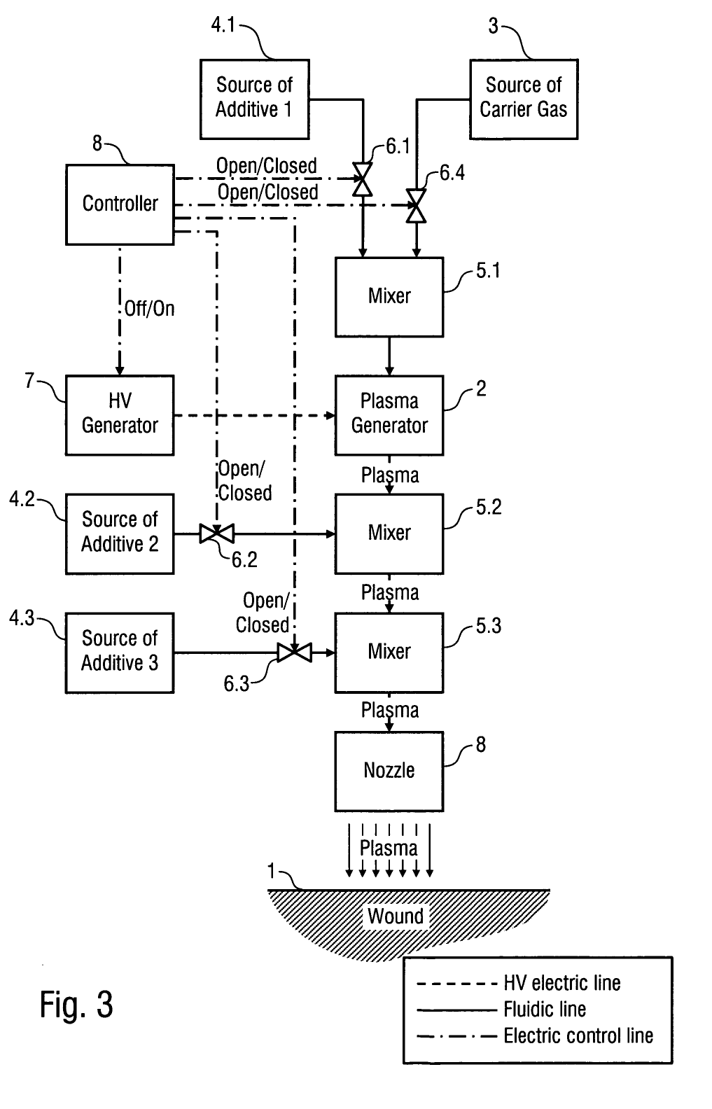

FIG. 3 shows a schematic representation of another embodiment of an apparatus for providing a non-thermal plasma wherein this apparatus is similar to the afore-mentioned apparatuses according to FIGS. 1 and 2 so that reference is made to the above description. Further, the same reference numerals are used for corresponding parts, components and details.

One characteristic of this invention is that the controller 8 actively controls the valves 6.1, 6.2. and 6.3. between the additive sources 4.1, 4.2, and 4.3 and the mixers 5.1, 5.2. and 5.3 and the high-voltage generator 7. The controller 8 also controls the valve 6.4. between the carrier gas source 3 and the mixer 5.1. The controller 8 synchronizes the valves 6.1., 6.4. and the high-voltage generator 7 in such a way that the time period during which the additive from the source 4.1. passes through the plasma generator 2 and the time period during which the carrier gas passes through the plasma generator 2 have a different temporal overlap with the time period during which the plasma generator 2 is activated. Therefore, the degrees of ionization (i.e. the percentage of the ionized atoms or molecules) of the additive from the source 4.1. and of the carrier gas are different and can be tuned independently of each other.

In this embodiment, two further additive sources 4.2, 4.3 are provided which are delivering different additives to mixers 5.2, 5.3 via valves 6.2, 6.3, wherein the mixers 5.2, 5.3 are arranged downstream behind the plasma generator 2 so that the plasma generator 2 does not ionize the additives provided by the additive sources 4.2, 4.3.

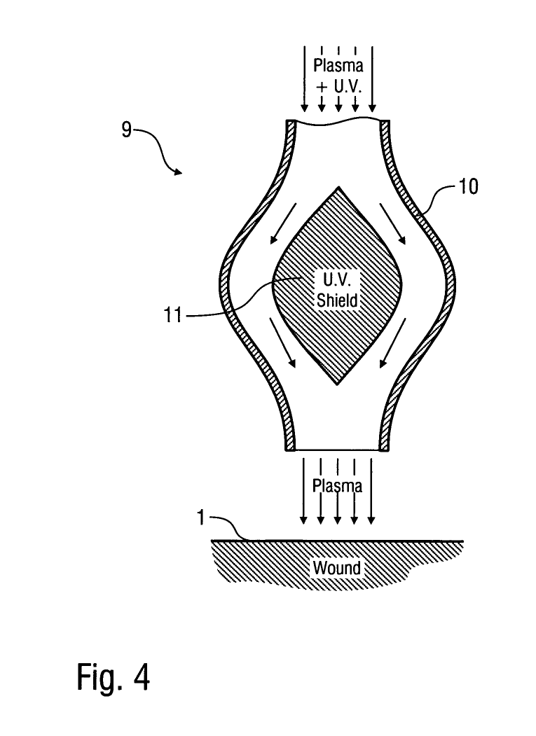

FIG. 4 shows a schematical representation of an outlet 9 of the apparatus according to the invention, wherein the outlet 9 applies the plasma to the wound 1.

The outlet 9 essentially consists of an outlet tube 10 guiding the plasma wherein ultraviolet radiation coming from the plasma generator enters the outlet tube 10, as well.

Therefore, the outlet 9 comprises a UV shield 11 which is arranged in the middle of the outlet tube 10 in a bulge of the outlet tube 10 so that the plasma flows around the UV shield 11. The UV shield 11 consists of a UV blocking material (e.g. regular window glass) and therefore blocks the ultraviolet radiation entering the outlet tube 10. Therefore, substantially no ultraviolet radiation leaves the outlet 9 so that the wound 1 is not affected by any ultraviolet radiation or only by a small fraction.

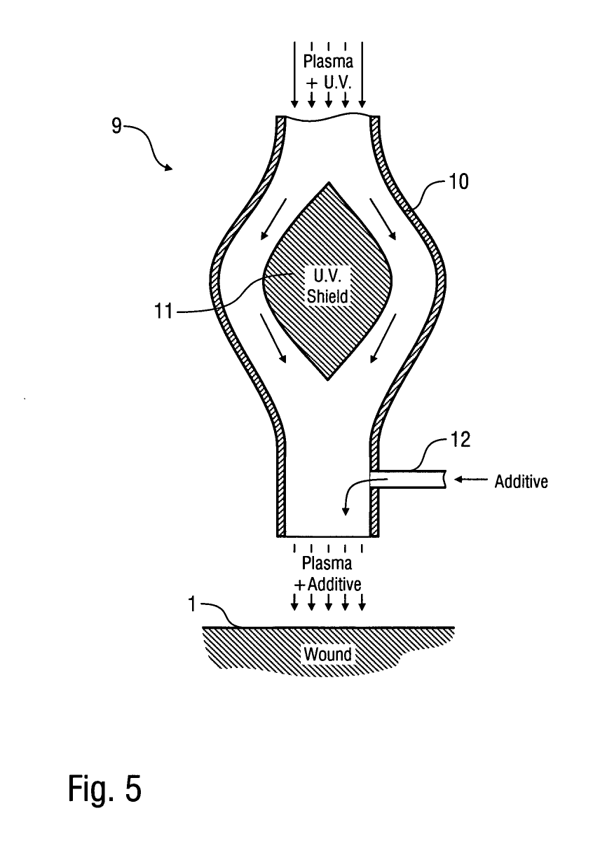

The embodiment of FIG. 5 is very similar to the embodiment of FIG. 4 so that reference is made to the above description with regard to FIG. 4. Further, the same reference numerals are used for corresponding parts, components and details.

One characteristic of this embodiment is that a conduit 12 discharges into the outlet tube 10 downstream behind the UV shield 11 wherein the conduit 12 delivers an additive to the plasma flow within the outlet tube 10. Therefore, the UV shield 11 prevents the additive from being affected by the ultraviolet radiation entering the outlet 9.

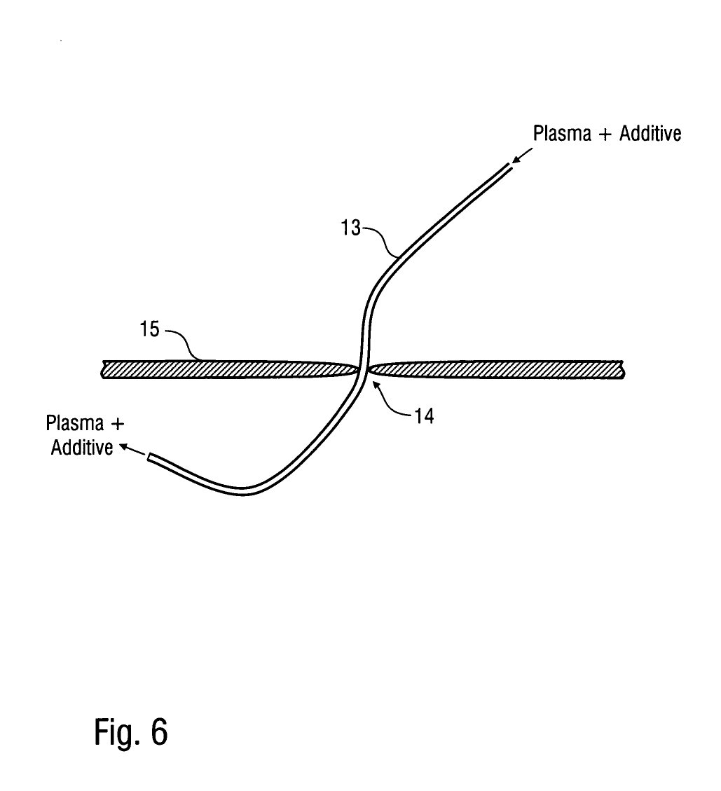

Finally, FIG. 6 shows a schematical representation of a hollow catheter 13 which can be inserted into a lumen of a human body through an artificial or natural body orifice 14 in body surface 15. For example, the body orifice 14 can be the mouth of a human being so that the catheter 13 is introduced into the gullet where the catheter 13 can apply the non-thermal plasma comprising the sterilizing additive to the gullet.

Although the invention has been described with reference to the particular arrangement of parts, features and the like, these are not intended to exhaust all possible arrangement of features, and indeed many other modifications and variations will be ascertainable to those of skill in the art.

### LIST OF REFERENCE NUMERALS

1 Wound
2 Plasma generator
3 Carrier gas source
4 Additive source
4.1 Additive source
4.2 Additive source
4.3 Additive source
5 Mixer
5.1 Mixer
5.2 Mixer
5.3 Mixer
6.1 Valve
6.2 Valve
6.3 Valve
6.4 Valve
7 High-voltage generator
8 Nozzle
9 Outlet
10 Outlet tube
11 U.V. shield
12 Conduit
13 Catheter
14 Body orifice
15 Body surface

1. Non-thermal plasma for treatment of a surface, wherein the plasma comprises a partially ionized carrier gas and at least one additive, which has a sterilizing effect on the surface.

2. Non-thermal plasma according to claim 1, wherein the additive is a member selected from the group consisting of:
a) Sulfates,
b) Chlorides,
c) Salts,
d) Metals,
e) Organic substances,
f) Inorganic substances,
g) Biomolecules,
h) Proteins,
i) Enzymes, and
j) Compounds or mixtures of the aforementioned substances.

3. Non-thermal plasma according to claim 1, wherein the additive is a member selected from the group consisting of:
a) Boron,
b) Bromine,
c) Thallium,
d) Silicon,
e) Iron,
f) Aluminium,
g) Silver,
h) Copper,
i) Zinc,
j) Manganese,
k) ZnSO₄,
l) K₂MnO₄,
m) FeSO₄,
n) Ti₂SO₄,
o) Iodine,
p) SiO₂,
q) KMnO₄,
r) Zinc Sulfate,
s) Copper(I) chloride or Copper(II) chloride,
t) Silver nitrate,
u) Silver chloride,
v) Manganese(II) sulfate,
w) (2-bromin-2-nitrovinyl)benzole,
x) Helium,
y) Neon,
z) Krypton,
aa) Xenon,
ab) Nitric oxide,
ac) Oxygen,
ad) Hydrogen,
ae) Sulfur hexafluoride,
af) Nitrous oxide,
ag) Hexafluorethane,
ah) Methane,
ai) Carbon fluoride,
aj) Fluoroform,
ak) Carbon dioxide,
al) Ethanol,
am) Air,
an) Water,
ao) Argon, and
ap) Compounds or mixtures of the aforementioned substances.

4. Non-thermal plasma according to claim 1, wherein the additive is a substance which can be either inactive or activated, and the plasma comprises the additive substantially in an inactive form.

5. A method of using the non-thermal plasma according to claim 1 for:
a) treating wounds,
b) treating living tissue,
c) treating organic tissue,
d) sterilizing a natural or artificial body orifice of a human or animal body,
e) sterilizing a medical instrument during insertion of the medical instrument through a body orifice into a lumen of a human or animal body,
f) sterilizing transplants,
g) treating skin diseases,
h) any medical treatment,
i) treating a visceral cavity or lumen of a human or animal body, or
j) manufacturing a medicine for treating wounds or biological tissue.

6. Apparatus for providing a non-thermal plasma, comprising:
a) a carrier gas source adapted for providing a carrier gas,
b) a plasma generator adapted for ionizing the carrier gas provided by the carrier gas source thereby generating the plasma,
c) an additive source providing an additive and
d) a mixer adapted for mixing the additive with one of the non-ionized carrier gas and the ionized plasma.

7. Apparatus according to claim 6, wherein the mixer is arranged upstream before the plasma generator and mixes the non-ionized carrier gas and the non-ionized additive, so that the plasma generator ionizes a mixture of the carrier gas and the additive.

8. Apparatus according to claim 6, further comprising:
e) several additive sources each providing a different additive, and
f) several mixers for mixing the different additives with one of the non-ionized carrier gas and the ionized plasma.

9. Apparatus according to claim 6, wherein
i) the plasma generator comprises an electrode arrangement or an antenna arrangement for electrically exciting the carrier gas thereby generating the plasma, and
ii) a high-voltage generator is connected to the electrode arrangement or the antenna arrangement.

10. Apparatus according to claim 9, wherein
iii) the mixer is arranged upstream before the plasma generator so that the plasma generator receives a mixture of the carrier gas and the additive, and
iv) the additive source provides the additive to the mixer discontinuously, so that there are additive-free intervals during which no additive is provided to the plasma generator.

11. Apparatus according to claim 29, further comprising a controller adapted to control the activation of the plasma generator and the gas flow from the additive source to the plasma generator in such a way that no additive is provided to the plasma generator during activation of the plasma generator.

12. Apparatus according to claim 9, wherein
iii) the high-voltage generator produces pulses which are separated by gaps, and
iv) the additive is provided to the plasma generator during the gaps only so that the additive is substantially not ionized.

13. Apparatus according to claim 9, wherein the electrode arrangement of the plasma generator is at least partially covered with a coating or comprises the additive so that the additive escapes from the coating into the carrier gas.

14. Apparatus according to claim 6, wherein
i) a UV shield is arranged between the plasma generator and an object to be treated so that any UV radiation emitted by the plasma generator is at least partially blocked by the UV shield and does not reach the object or only reaches a small fraction of the object, and
ii) the mixer is arranged downstream behind the UV shield so that the additive is added to the plasma downstream behind the UV shield and the additive is not affected by the UV radiation which is generated by the plasma generator.

15. Method of treating an object, comprising the following steps:
a) Providing a carrier gas,
b) Ionizing the carrier gas thereby generating a plasma,
c) Applying the plasma to the object, and
d) Mixing an additive with one of the carrier gas and/or the plasma before applying the plasma to the object.

16. Method according to claim 15, wherein the additive is mixed with the carrier gas upstream before the ionization.

17. Method according to claim 15, wherein the additive is mixed with the carrier gas upstream before the ionization of the carrier gas.

18. Method according to claim 17, further comprising the step of mixing several different additives with the carrier gas and/or with the plasma.

19. Non-thermal plasma according to claim 1, wherein
a) the non-thermal plasma is adapted for a treatment of a biological tissue, and
b) the non-thermal plasma has a beneficial effect on the biological tissue.

20. Non-thermal plasma according to claim 1, wherein
a) the non-thermal plasma is adapted for a treatment of a wound, and
b) the non-thermal plasma improves the healing of the wound.

21. Non-thermal plasma according to claim 1, wherein the additive is a substance which can be either inactive or activated, and the plasma comprises the additive substantially in an activated form.

22. Non-thermal plasma according to claim 1, wherein the additive is a substance which can be either dissociated or non-dissociated, and the plasma comprises the additive substantially in a dissociated form.

23. Non-thermal plasma according to claim 1, wherein the additive is a substance which can be either dissociated or non-dissociated, and the plasma comprises the additive substantially in a non-dissociated form.

24. Non-thermal plasma according to claim 1, wherein the additive is a substance which can be either coagulated or non-coagulated, and the plasma comprises the additive substantially in a coagulated form.

25. Non-thermal plasma according to claim 1, wherein the additive is a substance which can be either coagulated or non-coagulated, and the plasma comprises the additive substantially in a non-coagulated form.

26. Non-thermal plasma according to claim 1, wherein the additive is a substance which can be either ionized or non-ionized, and the plasma comprises the additive substantially in an ionized form.

27. Non-thermal plasma according to claim 1, wherein the additive is a substance which can be either ionized or non-ionized, and the plasma comprises the additive substantially in a non-ionized form.

28. Apparatus according to claim 6, wherein the mixer is arranged downstream behind the plasma generator and mixes the ionized carrier gas provided by the plasma generator and the non-ionized additive provided by the additive source.

29. Apparatus according to claim 10, wherein the plasma generator is activated during the additive-free intervals only.

30. Apparatus according to claim 10, wherein the ionization and the mixing of the additive temporally overlap so that the additive is partially ionized during the overlapping time period.

31. Apparatus according to claim 30, further comprising a controller adapted to control the activation of the plasma generator and the gas flow from the additive source to the plasma generator in such a way that the ionization and the mixing of the additive temporally overlap so that the additive is partially ionized during the overlapping time period.

32. Apparatus according to claim 9, wherein the additive source comprises a component comprising the additive or covered with the additive so that the additive escapes from the component, wherein the component is heatable to extract the additive from the component.

33. Apparatus according to claim 6, wherein a catheter is provided for introducing the plasma through a body orifice into a lumen of a human body.

34. Method according to claim 15, wherein the additive is mixed with the ionized carrier gas downstream behind the ionization so that the additive is not ionized.

35. Method according to claim 17, wherein the mixing of the additive and the ionization do not overlap temporally so that the additive is substantially not ionized.

36. Method according to claim 17, wherein the ionization and the mixing of the additive temporally overlap so that the additive is partially ionized during the overlapping time period.

37. Method according to claim 33, further comprising the step of introducing the plasma through a natural or artificial body orifice into a lumen of a human or animal body for treatment of the lumen.
